(19) United States
(12) Patent Application Publication
(10) Pub. No.: US 2015/0241855 A1
(43) Pub. Date: Aug. 27, 2015

(54) CONTROL SYSTEM FOR A CLEANING DEVICE, AND CLEANING DEVICE

(71) Applicant: OLYMPUS WINTER & IBE GMBH, Hamburg (DE)

(72) Inventor: Torben CARLSON, Hamburg (DE)

(73) Assignee: OLYMPUS WINTER & IBE GMBH, Hamburg (DE)

(21) Appl. No.: 14/707,731

(22) Filed: May 8, 2015

Related U.S. Application Data
(63) Continuation of application No. PCT/EP2013/003303, filed on Nov. 4, 2013.

(30) Foreign Application Priority Data
Nov. 13, 2012 (DE) 102012220617.5

(51) Int. Cl. G05B 11/01; G05B 15/02; A61B 19/00

(52) U.S. Cl. CPC G05B 11/01; A61B 19/34; G05B 15/02; A61B 2019/4894

(57) ABSTRACT

A control system for a cleaning device for cleaning surgical instruments, The control system including: a controlling or regulating device; at least one first sensor operatively connected to the controlling or regulating device, wherein the controlling or regulating device controls or regulates a cleaning processes of the cleaning device as a function of measured values of the at least one first sensor; a supplementary monitoring device; and at least one second sensor operatively connected to the supplementary monitoring device; wherein the supplementary monitoring device is electrically connected to the controlling or regulating device and is configured to exchange measured data and/or processed data of one or more of the at least one first sensor and the at least one second sensor between the controlling or regulating device and the supplementary monitoring device.

### CROSS-REFERENCE TO RELATED APPLICATION

The present application is a continuation of PCT/EP2013/003303 filed on Nov. 4, 2013, which is based upon and claims the benefit to DE 10 2012 220 617.5 filed on Nov. 13, 2012, the entire contents of each of which are incorporated herein by reference.

### BACKGROUND

1. Field

The present application relates to a control system for a cleaning device for cleaning surgical instruments, in particular endoscopes, with a controlling or regulating device, to which at least one first sensor is or can be attached, wherein the controlling or regulating device controls or regulates cleaning processes of the cleaning device as a function of measured values of the at least one first sensor. The application further relates to a cleaning device for cleaning surgical instruments, in particular endoscopes, with a controlling or regulating device by means of which cleaning steps of the cleaning device are monitored and controlled or regulated. The application also relates to a method for controlling or regulating the cleaning and/or treatment of surgical instruments, wherein a controlling or regulating device is provided, which processes measured data from a cleaning device received by means of at least one first sensor and uses it for controlling or regulating.

2. Prior Art

In cleaning devices or respectively treatment machines for surgical instruments, in particular endoscopes, a main controller for the monitoring of all installed sensors is currently used, wherein the cleaning steps are controlled and monitored by means of the main controller.

Such a cleaning device or respectively treatment device is thus known for example from EP 1 725 161 B1. A monitoring and control system for monitoring critical steps in a process for treating or respectively cleaning a medical device, which is executed by a hardware controller and the controller of a computer with a conventional operating system and with several inputs and outputs, which are connected with the hardware controller, is hereby used, wherein the monitoring and control system has a master controller, which is connected between the computer and the hardware controller and is set up to monitor critical steps in order to determine that each critical step is executed and executed correctly, and in order to take the controlling of the hardware controller away from the computer if an error is observed in a critical step.

A corresponding cleaning device or respectively treatment device, wherein a cleaning device is also to be understood as a treatment device for cleaning or respectively preparing surgical instruments, in particular endoscopes, provides for example to direct water through channels of the surgical instrument, for example of an endoscope, in order to rinse these channels in the surgical instrument. Additionally, chemical agents can be used to provide disinfection. Moreover, this process can take place under thermal action. Finally, the surgical instruments are also correspondingly cleaned from outside. Moreover, it is normally provided to check whether the channels of the surgical instrument are sealed or plugged. Finally, it can also be provided to remove the water or respectively aqueous solutions from the surgical instrument with compressed air.

The flexibility, i.e. the adaptability to different requirements, of corresponding cleaning devices is limited. Thus, it takes much effort for example to react for example to country-specific requirements, which makes the cleaning devices unnecessarily expensive to produce.

### SUMMARY

An object is to increase the flexibility of a corresponding cleaning device and to specify a control system for a cleaning device for cleaning surgical instruments, in particular endoscopes, as well as a cleaning device for cleaning surgical instruments and a method for controlling or regulating the cleaning and/or treatment of surgical instruments, which enables increased flexibility.

This object is solved by a control system for a cleaning device for cleaning surgical instruments, in particular endoscopes, with a controlling or regulating device, to which at least one first sensor is or can be attached, wherein the controlling or regulating device controls or regulates cleaning processes of the cleaning device as a function of measured values of the at least one first sensor, which is further developed in that a supplementary monitoring device is provided, to which at least one second sensor is or can be connected, wherein the supplementary monitoring device is or can be electrically connected to the controlling or regulating device and is designed to exchange measured data and/or processed data of the at least one first sensor and/or second sensor between the controlling or regulating device and the supplementary monitoring device.

By providing a supplementary monitoring device, for one, increased flexibility is possible, since additional sensors can thus be used, which are not or do not need to be provided in a base control system. Thus, for example, a base control system can be provided, which provides the controlling or respectively regulating of the cleaning device, which is necessary and required in some countries. For countries with additional requirements, a corresponding supplementary monitoring device with at least one second sensor can then be provided. Moreover, increased reliability of the cleaning process, which is executed with the control system, results from the solution according to the present disclosure.

The data exchange can serve to control or regulate the cleaning device. For example, data from the at least one second sensor can hereby serve to correspondingly modify the cleaning processes in the controlling or regulating device.

The data exchange can serve to monitor the controlling or regulating device through the supplementary monitoring device. It is hereby ensured that the control system provides a very secure cleaning or respectively treatment.

A documentation device can be attached to the supplementary monitoring device or the supplementary monitoring device has a documentation device. It is hereby possible to document all relevant data of the control system, i.e. both data from the at least one first sensor and control data from the controlling or regulating device as well as data from the at least second sensor and from the supplementary monitoring device.

Accordingly, the documentation device can document data from the supplementary monitoring device and the controlling or regulating device.

The object is also solved by a cleaning device for cleaning surgical instruments, in particular endoscopes, with a controlling or regulating device, by means of which cleaning steps of the cleaning device are monitored and controlled or regulated, wherein the cleaning device is designed to accommodate or attach at least one supplementary monitoring device, which is or can be connected with the controlling or regulating device, wherein the supplementary monitoring device executes further monitoring functions and is designed to exchange data with the controlling or regulating device. At least one first sensor can be hereby connected with the controlling or regulating device. Furthermore, at least one second sensor can be connected with the supplementary monitoring device. The data exchange can serve to control or regulate the cleaning device. Furthermore, the data exchange can serve to monitor the controlling or regulating device through the supplementary monitoring device.

A supplementary monitoring device can be provided for use in a control system or cleaning device. Furthermore, a supplementary monitoring device can be used in a control system or a cleaning device.

The object is further solved by a method for controlling or regulating the cleaning and/or treatment of surgical instruments, wherein a controlling or regulating device is provided, which processes measured data recorded by means of at least one first sensor from a cleaning device and uses it for controlling or regulating, wherein furthermore measured data from the cleaning device is also captured by at least one second sensor and processed by a supplementary monitoring device, wherein the measured data processed by the supplementary monitoring device serves to monitor the controlling or regulating device and/or is directed to the controlling or regulating device for processing for the controlling or regulating. The method ensures a high process reliability for the cleaning or respectively treatment of surgical instruments. Moreover, the method can be adapted very flexibly to the needs of the user.

The at least one first sensor can measure at least one temperature, at least one pressure and/or at least one flow rate in the cleaning device. The flow rate usually means the flow rate of a channel of the surgical instrument or respectively endoscope. Accordingly, the pressure can be measured outside the surgical instrument and/or within the surgical instrument or respectively a supply line to a channel of the surgical instrument. The temperature can also be measured in a supply line to a channel of the surgical instrument or outside the surgical instrument in a space, into which the surgical instrument is mounted, for example, in a liquid bath.

The at least one second sensor can measure at least one temperature, at least one pressure, at least one flow rate, at least one inductivity, at least one pH value and/or at least one contamination level. The temperature, the pressure and the flow rate can be the same measurement parameters or respectively measurement variables as those measurement variables measured by the at least one first sensor. Different first sensors and second sensors can be used for the different variables. Moreover or in addition or alternatively, the at least one second sensor can measure an inductivity, a pH value and/or at least one contamination level. The inductivity or respectively the pH value or the contamination level are measurement variables of the used liquid or respectively liquids for cleaning or treatment of the surgical instrument.

The supplementary monitoring device can end the cleaning and/or treatment of the surgical instrument and/or the supplementary monitoring device emits a fault signal if, for a specifiable value for a measurement value of the at least one second sensor, or in the case that the at least one first sensor and the at least one second sensor measures the same variable, in the case of a specifiable deviation from it. Thus, if for example the contamination level lies above a contamination level threshold value, a fault signal can be emitted or the cleaning and/or treatment of the surgical instrument can be ended. Alternatively, the liquid can also be pumped out for this and a clean liquid can be used for the process. The contamination level can be performed for example via an optical measurement. There can also be functionality whereby the supplementary monitoring device ensures in the case of corresponding recorded measurement values that for different values, which are measured by the first sensor for the same variable as for the second sensor, the supplementary monitoring device ends the cleaning and/or treatment of the surgical instrument or emits a fault signal. Accordingly, it can also be provided that, in this case, the controlling or regulating device ends the cleaning and/or treatment process and/or emits a fault signal.

The supplementary monitoring device can log the measured data of the at least one first sensor and of the at least one second sensor, for example by providing a documentation device in or on the supplementary monitoring device or by transmitting the measured data to a documentation device to be provided separately from the supplementary monitoring device or respectively the cleaning device.

Further characteristics will become evident from the description of the embodiments together with the claims and the attached drawing. The disclosed embodiments can fulfill individual characteristics or a combination of several characteristics.

### BRIEF DESCRIPTION OF THE DRAWINGS

The embodiments are described below, without restricting the general idea of the invention, based on an exemplary embodiment with reference to the drawing, whereby we expressly refer to the drawing with regard to the disclosure of all details according to the invention that are not explained in greater detail in the text. In the FIGURE:

The FIGURE illustrates a schematic overview of a control system for a cleaning device.

### DETAILED DESCRIPTION

The FIGURE shows a schematic block diagram for a control system. A cleaning device 10, in which a first sensor 13 and a second sensor 14 are accommodated, is indicated by the dashed line. The respective sensors serve to record measurement variables or respectively parameters in the cleaning device. These can be the temperature, a flow rate, a pressure, an inductivity, a pH value and/or a contamination level. The at least one first sensor 13, which is shown, and the at least one second sensor 14, which is also shown, can also be a plurality of sensors, which record different measurement variables.

The at least one second sensor 14 can record completely or partially the measurement variables, which the at least one sensor 13 records and also lie mainly at the same location; i.e. if for example an endoscope with six channels, which are to be cleaned and rinsed, is provided, a first sensor 13 can be provided for each channel and a second sensor 14 in order to measure for example the flow rate or the pressure there. A very secure and reliable operation or respectively a very secure and reliable cleaning or treatment of the surgical instrument, in this case the endoscope, is then possible through the double measurement. The at least one second sensor 14 can however also measure different measurement variables than the at least one first sensor 13 or measure both the same as well as other measurement variables. Several second sensors 14 are then provided for this.

The measured data of the at least one first sensor 13 are supplied to the controlling or regulating device 11 via a data connection 15. This controls or regulates cleaning and/or treatment processes in the cleaning device 10. A supplementary monitoring device 12 is provided, which is connected with data connections 17 and 18 with the controlling or regulating device 11. The data connection 17 serves to transmit data from the controlling or regulating device 11 to the supplementary monitoring device 12 and the data connection 18 serves to transmit data from the supplementary monitoring device 12 to the controlling and regulating device 11. The supplementary monitoring device 12 receives measured data from the at least one second sensor 14 via a data connection 16. The data connections shown here can be either wired and wireless. A bidirectional data connection can also be provided between the controlling or regulating device 11 and the supplementary monitoring device 12.

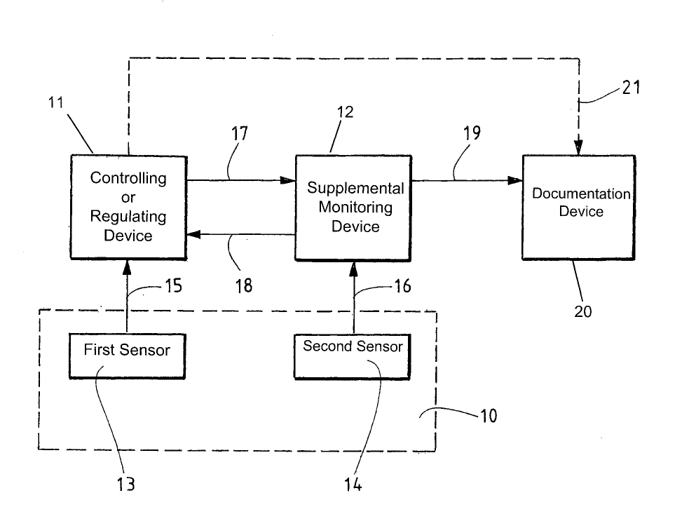

Data from the controlling or regulating device 11 and also from the supplementary monitoring device 12 can be transmitted to a documentation device 20 via a data connection 19, which is provided externally to the supplementary monitoring device 12 in the exemplary embodiment according to FIG. 1. The documentation device 20 can however also be integrated in the supplementary monitoring device 12. The controlling or regulating device 11 can also transmit data directly to the documentation device 20 via a data connection 21, for example in order to be able to monitor the correct functionality of the supplementary monitoring device 12. The data connections can be performed via normal interfaces, for example via a CAN bus or via a USB interface. Other interfaces are also conceivable.

In order to achieve greater flexibility than previously usual, it is now possible to provide a cleaning device both with an interface to a main controller in the form of a controlling or regulating device 11 and to also attach additional sensors to a supplementary monitoring device 12 and to make available the evaluation results to the supplementary monitoring device 12 with the main controller or respectively the controlling or regulating device 11. Moreover, it is possible to provide country-specific sensors, i.e. to provide sensors in a flexible manner, i.e. to not provide them in a basic version of the cleaning device, and to then use these second sensors by means of the supplementary monitoring device 12 in order to record and to process corresponding measured data. A simple and cost-effective as well as flexible retrofitting is thus possible for the different countries. Moreover, a very reliable documentation of the cleaning and treatment processes is possible. The documentation can document both data from the sensors of the controlling or regulating device 11 as well as data from the sensors of the supplementary monitoring device 12.

Via the data connection 17, the controlling or regulating device 11 can also transmit machine data to the supplementary monitoring device 12, which has a further monitoring function of this machine data. Should for example faults occur, this can be determined via the supplementary monitoring device 12 and action can be taken via the data connection 18.

All named characteristics, including those taken from the FIGURE alone, and individual characteristics, which are disclosed in combination with other characteristics, are considered alone and in combination as essential for the invention. Embodiments according to the invention can be realized by individual characteristics, or a combination of several characteristics.

### LIST OF REFERENCE NUMBERS

10 Cleaning device
11 Controlling or regulating device
12 Supplementary monitoring device
13 First sensor
14 Second sensor
15 Data connection
16 Data connection
17 Data connection
18 Data connection
19 Data connection
20 Documentation device
21 Data connection

What is claimed is:

1. A control system for a cleaning device for cleaning surgical instruments, the control system comprising:
a controlling or regulating device;
at least one first sensor operatively connected to the controlling or regulating device, wherein the controlling or regulating device controls or regulates a cleaning processes of the cleaning device as a function of measured values of the at least one first sensor;
a supplementary monitoring device; and
at least one second sensor operatively connected to the supplementary monitoring device;
wherein the supplementary monitoring device is electrically connected to the controlling or regulating device and is configured to exchange measured data and/or processed data of one or more of the at least one first sensor and the at least one second sensor between the controlling or regulating device and the supplementary monitoring device.

2. The control system according to claim 1, wherein the data exchange serves to control or regulate the cleaning device.

3. The control system according to claim 1, wherein the data exchange serves to monitor the controlling or regulating device by the supplementary monitoring device.

4. The control system according to claim 1, further comprising a documentation device one of operatively connected to the supplementary monitoring device or contained in the supplementary monitoring device.

5. The control system according to claim 4, wherein the documentation device documents data from the supplementary monitoring device and the controlling or regulating device.

6. A cleaning device for cleaning surgical instruments the cleaning device comprising:
a controlling or regulating device by which cleaning steps of the cleaning device are monitored and controlled or regulated;
at least one supplementary monitoring device operatively connected to the controlling or regulating device,
wherein the supplementary monitoring device executes further monitoring functions and is configured to exchange data with the controlling or regulating device.

7. The cleaning device according to claim 6, wherein the data exchange serves to control or regulate the cleaning device.

8. The cleaning device according to claim 6, wherein the data exchange serves to monitor the controlling or regulating device by the supplementary monitoring device.

9. A method for controlling or regulating the cleaning and/or treatment of surgical instruments, the method comprising:
using a first controller to process first measured data from a cleaning device and recorded by at least one first sensor;
using a second controller to process second measured data from the cleaning device and recorded by at least one second sensor; and
wherein the second measured data processed by the second controller serves to one or more of monitor the first controller or is directed to controlling or regulating the first controller.

10. The method according to claim 9, wherein the at least one first sensor measures at least one of temperature, pressure and flow rate in the cleaning device.

11. The method according to claim 9, wherein the at least one second sensor measures at least one of temperature, pressure, flow rate, inductivity, pH value and contamination level.

12. The method according to claim 9, wherein the second controller, for a specifiable value for a measurement value of the at least one second sensor, or in the case that the at least one first sensor and the at least one second sensor measures the same variable, in the case of a specifiable deviation from the specific value, one or more of ends the cleaning and/or treatment of the surgical instrument and emits a fault signal.

13. The method according to claim 9, wherein the second controller logs the first measured data of the at least one first sensor and the second measured data of the at least one second sensor.
